(19) United States
(12) Patent Application Publication
(10) Pub. No.: US 2023/0070796 A1
(43) Pub. Date: Mar. 9, 2023

(54) METHOD FOR EVALUATING RESULTS OF AN AUTOMATIC CLASSIFICATION

(71) Applicant: COLLECTIVE THINKING, Paris (FR)

(72) Inventors: Vincent SUSPLUGAS, Paris (FR); Edouard DUFETELLE, Paris (FR)

(73) Assignee: COLLECTIVE THINKING, Paris (FR)

(21) Appl. No.: 17/794,879

(22) PCT Filed: Jan. 22, 2021
(86) PCT No.: PCT/EP2021/051518

§ 371 Date: Sep. 28, 2022

(30) Foreign Application Priority Data
Jan. 24, 2020 (FR) 2000730

(51) Int. Cl. G16H 40/20; G16H 10/60

(52) U.S. Cl. CPC G16H 40/20; G16H 10/60

(57) ABSTRACT

The invention relates to a method (100) for evaluating results of an automatic classification of medical data (7), comprising the implementation of the following steps:
computer calculating means (4) perform an automatic classification of one or more medical data (7),
the computer calculating means (4) determine a confidence level (9, 20) associated with at least one result (8) of the classification,
the computer calculating means (4) compare the confidence level (9) associated with the result with a predetermined confidence threshold, and
the computer calculating means (4) display the result to a user (2) if the confidence level associated with the result is higher than or equal to the predetermined confidence threshold.

The invention relates to the evaluation of automatic classification proposals, especially in the medical field. In particular, it relates to the evaluation of the automatic classification of medical stays within classes of diagnoses and medical acts.

The healthcare establishments are obliged to produce the PMSI (Program of Medicalisation of Information Systems) encoding. As part of this encoding, for each medical stay of a patient, the medical diagnoses of the patient and any acts or procedures performed during the stay are described and documented: the classification code(s) of the diagnoses and acts or corresponding procedures are therefore associated with each stay. These data are generated for the purposes of epidemiological research, analysis of the hospital activity and hospital funding.

In particular, the main method of funding healthcare establishments is activity-based pricing. This pricing is based on measuring the type and volume of the activities. Consequently, each medical stay of a patient in a healthcare establishment is classified in particular within a “Diagnosis Related Groups” (DRG) which is associated with a “Homogeneous Group of Stays” (HGS). This classification determines the price of the hospital stay covered by the health insurance schemes.

This classification activity is carried out by the medical information departments of the healthcare establishments. They must perform an in-depth analysis of the clinical record of each patient after their medical stay to extract the relevant information in order to determine to which code, i.e. in which DRG/HGS class, this stay must be assigned. It is a tedious and complex activity presenting a high risk of errors likely to have a significant impact on the hospital income, the epidemiological research or any other type of analysis of these data.

A method for assisting PMSI encoding by automatic classification is already known in the state of the art. According to this method which involves automatic learning technologies, a “self-learning” model is trained to classify each medical stay automatically. For each stay, therefore, medical data corresponding to this stay, such as the clinical record of the patient, are supplied as input to the trained model which, according to these data and its learning, assigns diagnosis codes and act or procedure codes, or even an DRG/HGS code directly, to the stay. This method is used to automatically classify the stays for which there is very little doubt concerning the classification. It concerns for example stays for which the diagnosis is quite obvious from the clinical record. It also concerns stays for which, in view of the large number of stays with similar medical data on which the model is trained, there is no doubt for the model regarding the assignment of a particular code.

However, this method is not capable of automatically classifying all the stays with a sufficient confidence level. In particular, the classification of some stays is more difficult due for example to the complexity of the medical record or the nature of some medial data supplied as input. In other words, this concerns the stays for which prior automatic training is not sufficient to ensure that the model will perform a classification without a high risk of errors. For these stays, the error rate of an automatic classification is too high for the learning model to classify them automatically. For each of these stays which are more difficult to classify, this model of the method of the state of the art can provide classification proposals to a human user, but there are numerous proposals which are generally not precise enough to allow automatic classification of the most probable proposal. The user must therefore consult the medical record in order to choose the right code for each of these stays. Once again therefore, these tasks are tedious and complex.

The invention aims in particular to simplify and accelerate the classification of medical data that a learning model cannot classify automatically without a high risk of error.

Thus, the invention relates to a method for evaluating results of an automatic classification of medical data, comprising the implementation of the following steps:
computer calculating means perform an automatic classification of one or more medical data,
the computer calculating means determine a confidence level associated with at least one result of the classification,
the computer calculating means compare the confidence level associated with the result with a predetermined confidence threshold, and
the computer calculating means display the result to a user if the confidence level associated with the result is higher than or equal to the predetermined confidence threshold.

Instead of “result”, we may also speak of “result proposal” made by the calculating means after the classification step. The result(s) must then be validated by the user, in other words displayed on screen, only if they correspond to an acceptable confidence level which is configured by the user. A “result” type is the class to which a learning model proposes to assign a medical data or a set composed of medical data, supplied as input. By “confidence level”, we may speak in particular of “precision” in the statistical meaning of the term. Lastly, in the remainder of the application, we will speak indifferently of “classification” or “labelling”, which are two terms that have the same definition in this context: assign as output one or more classes to a medical data or to a set of medical data supplied as input. This concerns in particular classification proposals whose confidence level is not high enough to allow automatic classification, but high enough for the results proposed to be examined and validated quickly by the user, if necessary. The user can therefore decide to display, for sets of input data, all the results proposed for each input corresponding to an acceptable confidence level, and decide not to display the results which do not correspond to this confidence level. Consequently, the user avoids automatic classification of results for which the risk of error remains high, and at the same time can, for these results, simply perform a quick check to determine whether the class proposed as output for a given input seems to be the right one. As a corollary, by proposing this intermediate “quasi-automation” step, this method limits the in-depth study of medical data to those whose prediction is not precise enough to simply perform a quick check of the proposed result. Thus, the method accelerates and simplifies the classification of the medical data which it is too risky to classify automatically, by having the relevant results validated by the user. The user can concentrate on these data for which automatic classification would be risky, while benefiting from a probable result proposal. The work is therefore easier and quicker to perform for the user, who therefore makes fewer classification errors than if they had to perform an in-depth analysis of all the data not classified automatically. Lastly, the term “computer calculating means” designates any computer device or sets of devices, such as a processor, a memory, a computer or even a complete set of servers, used to numerically process data upon command or automatically. In the application, we also speak indifferently of “automated means”.

The confidence level determined can be associated with the result directly, for example if it is associated directly with the class proposed as output for the medical data supplied as input. However, it may also be associated with the result indirectly, for example if it is not associated with the proposed class, but with a larger class including the proposed class as well as other classes.

Preferably, the automated means also display to the user at least one detected justification data justifying the result.

Thus, in addition to viewing only the most probable results, the user can also view at least one justification data associated with the result. This data is an indication allowing the user to check at a glance whether the result proposed is the right one for the medical data corresponding to this result.

This characteristic further accelerates the classification of medical data, since the user no longer has to search in their data, for example in a medical record, to check whether or not the result proposed is the right one, but can simply consult the one or more justification data to decide whether or not the result proposed is in fact the right one.

Advantageously, the justification data belongs to the one or more data for which the classification proposal performed led to the result, and is textual.

Thus, the computer calculating means find, amongst the medical data being classified, a word or sentence allowing the user to determine whether the corresponding result seems adequate. This word or sentence is the justification data. It may be, for example, a medical term relating to medical care, a diagnosis or a medical symptom.

Preferably, in addition,
the computer calculating means also determine a justifiability level associated with the result,
the computer calculating means compare the justifiability level associated with the result with a predetermined justifiability threshold,
the computer calculating means display the result to the user if, in addition, the justifiability level is higher than or equal to the predetermined justifiability threshold.

Thus, the confidence level is not the only criterion considered to display or not a result to the user, as the justifiability level is also considered. It corresponds to a quantity or quality of data allowing the user to justify the proposed result. In other words, to be displayed, the result must not only have a confidence level higher than the predetermined confidence threshold, but in addition the calculating means determine whether they can display to the user convincing data associated with this result, so that the user can quickly make a classification decision. Thus, according to this embodiment, even if a proposed result is probably the right one, if the calculating means are unable to supply data allowing the user to validate this result easily, they do not display it to the user. The user can therefore concentrate on the proposed results which are both probable and justified. The results displayed are therefore even more relevant since their justifiability allows the user to validate them more easily.

As for the confidence threshold, the justifiability threshold can be configured by the user as required.

Advantageously, to determine the justifiability level associated with the result:
amongst the one or more medical data, the computer calculating means detect at least one or one of the justification data justifying the result, and
depending on the or each justification data detected, the computer calculating means determine the justifiability level associated with the result.

Thus, the justifiability level depends on the number and/or the relevance of the justification data detected by the automated means amongst the medical data supplied as input. The greater the number and/or relevance of these justification data, the higher the justifiability level since the calculating means can display to the user justification data which will considerably help the user to validate the result.

Preferably, to detect a justification data justifying the result, the computer calculating means first determine one or more types of justification data to be detected.

Thus, the self-learning model is trained to detect justification data on a learning database.

Advantageously, to determine one or more types of justification data to be detected, from a learning medical database:
using learning medical data from the database, the computer calculating means perform a first classification and determine a first confidence level associated with a result of the first classification,
the computer calculating means mask at least one of the learning medical data used to perform the first classification,
the computer calculating means perform a second classification using data not including the masked learning medical data and determine a second confidence level of the second classification,
if the difference between the first and second confidence levels is higher than a predetermined threshold, the computer calculating means record a type of the masked learning medical data as type of justification data to be detected.

Thus, to determine the classification data to be detected, some of the medical data are removed or masked during the learning and the impact of removing these data on the confidence level output from the classification is measured. If the confidence level has dropped considerably, this means that the medical data which were masked are especially important to determine the class corresponding to the stay to which these data belong. These masked data are therefore considered as justification data which can be displayed to the user if they are detected, to accompany and justify the proposed result. This method is therefore performed before classifying the medical data under “real conditions” to determine the types of data to be detected. In addition, since it can be repeated for input training data that are different but correspond to identical results, the means determine the most common justification data types associated with each possible result, irrespective of the data supplied as input.

As an alternative, to detect a justification data justifying the result, one or more types of justification data to be detected are supplied to the computer calculating means by the user.

Thus, in addition to or instead of the justification data being determined by the computer calculating means, a human user can supply to the means a list of justification data to be detected, for example the words the user deems relevant. Once again, the data are supplied to the means before the classification.

As an alternative, the computer calculating means detect the one or more justification data using the one or more medical data for which the automatic classification performed led to the result.

Thus, the type(s) of justification data to be detected are determined this time a posteriori, in other words when the classification has been performed. This method avoids the need to determine beforehand types of justification data for all possible results.

Preferably, to detect the one or more justification data using the one or more medical data for which the automatic classification performed led to the result, since the classification is a first classification and the confidence level associated with the result is a first confidence level:
the computer calculating means mask the or at least one of the medical data used to perform the first classification,
the computer calculating means perform a second classification using the data not including the masked medical data and determine a second confidence level of the second classification,
if the difference between the first and second confidence levels is higher than a predetermined threshold, the computer calculating means (4) record the masked medical data as detected justification data.

Thus, the means implement a method similar to that used to detect a priori types of detection data and described above, in other words through the use of classifications with masked data, but in this case it is performed after the classification and only for the proposed result.

Preferably, the result belongs to a tree structure of nodes corresponding to possible results.

Thus, the class to be assigned as output to the data supplied as input may belong to a tree structure of classes arranged together hierarchically. In other words, a “parent” node of the tree structure corresponds to a class including several sub-classes, themselves associated respectively with “son” nodes of the parent node. A result may correspond to any node, in other words to any hierarchic level in the tree structure of classes. Such a level may in fact be configured by the user, who can decide to display only the results of a certain hierarchic level. Generally in fact, the higher the hierarchic level, the lower the risk of error in the classification, but the less the class assigned is adapted to the data supplied as input since it is a more generic class.

Advantageously, the confidence level is associated with a node of the tree structure which is a parent of a node corresponding to the result.

Thus, the proposed result corresponds to a precise class, but the confidence level is determined for a parent class, in other words more generic and comprising the class corresponding to the result. The user can therefore display precise results, but relying on a confidence level associated with a hierarchic level that is more generic than that of the results. In concrete terms, results which would not be displayed if the confidence level was associated directly with the result are displayed in this case since they correspond to sister classes or classes close to the result. The user therefore extends the result display criteria to classes close to the correct class, but without necessarily modifying a confidence threshold. The method can therefore be used in this case to display to the user results that are at least close, in terms of classification, to the correct results, even if the precision of these results is not sufficient for them to be displayed normally.

As an alternative, the confidence level is associated with a group of nodes of the tree structure comprising a node corresponding to the result.

Thus, it is not the confidence associated with the proposed result which is determined in this case, but that granted to a group of possible results. In other words, it is the confidence granted to the fact that one of the members of the group is the correct result.

Advantageously, the justifiability level is associated with a node of the tree structure which is a parent of a node corresponding to the result.

As an alternative, the justifiability level is associated with a group of nodes of the tree structure comprising a node corresponding to the result.

These two options correspond to what was mentioned above, but applied this time to the justifiability level.

Preferably, depending on the confidence level associated with the result, the computer calculating means modify the association of the confidence level and preferably the confidence threshold.

Thus, the computer calculating means automatically adapt the hierarchic level with which the confidence level is associated, or even the confidence threshold to be reached, depending on the result. In other words, if for a stay, no result can initially be displayed to the user since the confidence threshold has not been reached, the computer calculating means “return” to a parent or brother node, group of nodes or node close to the node corresponding to the result, and determine whether they find therein another result reaching the possibly modified confidence threshold. The user therefore no longer needs to adapt the adjustment of their confidence level each time the results are evaluated, since the computer calculating means perform this adaptation in order to display a result for each set of medical data supplied as input, even if the hierarchic level of this result or the confidence level with which it is associated does not correspond to the levels and thresholds targeted initially.

Similarly, advantageously, depending on the justifiability level associated with the result, the computer calculating means modify the association of the justifiability level and preferably the justifiability threshold.

Preferably, the computer calculating means allow a user to predetermine the confidence threshold and/or the justifiability threshold.

The user can configure these thresholds using precision-sensitivity curves. The more demanding the user is regarding the precision levels of these thresholds, the more the results displayed will be relevant, but the risk that no result is displayed for a given stay will also be higher. Inversely, the lower a precision level, the higher the risk of displaying an incorrect result for a given stay.

Advantageously, the computer calculating means allow the user to reject the result.

Thus, in this case the method validates the result proposed by the computer calculating means, but gives the user the opportunity to cancel this validation. Consequently, if most of the results are correct, the user simply needs to act on the other minority classifications, in other words those which turned out to be incorrect.

Preferably, the or at least one of the medical data concerns at least one medical stay of at least one patient in a healthcare establishment.

Thus, the method can be used in particular to improve the computer classification of the medical stays, in particular to improve the accounting precision of the healthcare establishments, to perform the epidemiological researches or any other type of analysis of these medical stays.

Advantageously, since several medical data concern several medical stays, the computer calculating means display to the user the classification results of all the stays.

Thus, the user can very quickly consult the results for all the stays they want to classify. In other words, the user does not have to navigate between several results and can quickly decide whether to validate or reject a list of results.

Preferably, the proposal result corresponds to one or more codes concerning medical diagnoses, acts or procedures, Diagnosis Related Groups (DRG) or Homogeneous Groups of Stays (HGS).

It may correspond in particular to codes, ICD10, COMA or DRG/HGS relating to the PMSI encoding. The method therefore simplifies and accelerates the PMSI encoding procedure in healthcare establishments, in particular for medical stays for which the risk of incorrect classification is too high. The medical information departments of these establishments can therefore decide on the classification and therefore the encoding, by relying on the most probable results supplied by the self-learning model for a list of stays, without having to perform an in-depth study of the medical data corresponding to each stay.

Advantageously, the confidence level associated with the result is a numerical value associated with a probability that the result is correct.

Thus, the confidence level corresponds to the percentage chance that the proposed result is correct.

Preferably, to determine the numerical value, using a learning medical database:
the computer calculating means classify learning data;
for each classification, the computer calculating means determine primary numerical values associated respectively with classification results;
for each classification, the computer calculating means select the classification result having the highest numerical value;
for each classification, the computer calculating means compare the selected result with a correct expected result; and
depending on the comparison results, the computer calculating means determine the numerical value associated with a probability that the selected result is correct.

Thus, the confidence level calculated for each stay corresponds to two elements:

the proposed result and the primary numerical value assigned to it during the classification. The model is trained to determine a precision level for combinations of these two elements. Then, whenever a result is proposed with a certain primary numerical value, the model can statistically deduce a degree of precision, in other words the confidence level associated with this result and this primary value.

The invention also relates to a data processing system comprising means for implementing the steps of the method described previously.

The invention also relates to a computer program, comprising instructions which, when the program is executed by a computer, instruct the computer to implement the steps of the method described previously.

The invention also relates to a method of obtaining the preceding program in order to download it on a telecommunication network.

The invention also relates to a computer-readable data medium, on which the computer program described previously is stored.

### BRIEF DESCRIPTION OF THE FIGURES

The invention will be better understood on reading the following description, given solely by way of example and with reference to the accompanying drawings in which:

FIG. 1 shows the means implemented in the invention;

FIG. 2 shows the results evaluated by the invention;

FIG. 3 shows a conventional method for configuring an automatic classification model;

FIG. 4 shows a method for determining a confidence level;

FIG. 5 shows a method for determining a justifiability level;

FIG. 6 shows a first variant of a step of the method of FIG. 5;

FIG. 7 shows a second variant of a step of the method of FIG. 5;

FIG. 8 shows an example of a precision-sensitivity curve;

FIG. 9 shows the progression of a user interface;

FIG. 10 shows a first embodiment of the invention;

FIG. 11 shows a second embodiment of the invention.

### DETAILED DESCRIPTION

The context of the invention is the obligation for healthcare establishments to identify, for each stay, the diagnoses and acts characteristic of the patient. For budgetary reasons in particular, these establishments must report each medical stay of a patient by assigning “PMSI” codes to said stay. Since each code corresponds to a main medical diagnosis and possibly to one or more associated diagnoses as well as to one or more medical acts, the competent bodies such as the social security can reimburse the hospitals on the basis of these codes.

An automatic classification method is used to assign one or more PMSI codes to each medical stay. It uses an input a set of data concerning a medical stay and supplies as output a PMSI code which, in the best case, correspond to the stay, but which is sometimes incorrect or insufficiently precise.

The invention does not relate to automatic classification as such, but to an evaluation of the results of an automatic classification, in other words evaluating whether or not the right code has been assigned to the right stay. This also explains why the invention is not limited to a particular automatic classification method or model. On the contrary, it can be used to evaluate the results of automatic classification proposals produced by different models.

We will first describe all the means implemented as well as all the technical steps required to understand and implement the invention. We will then summarise these elements by describing the embodiments of the invention.

I. The Means

A) The Elements

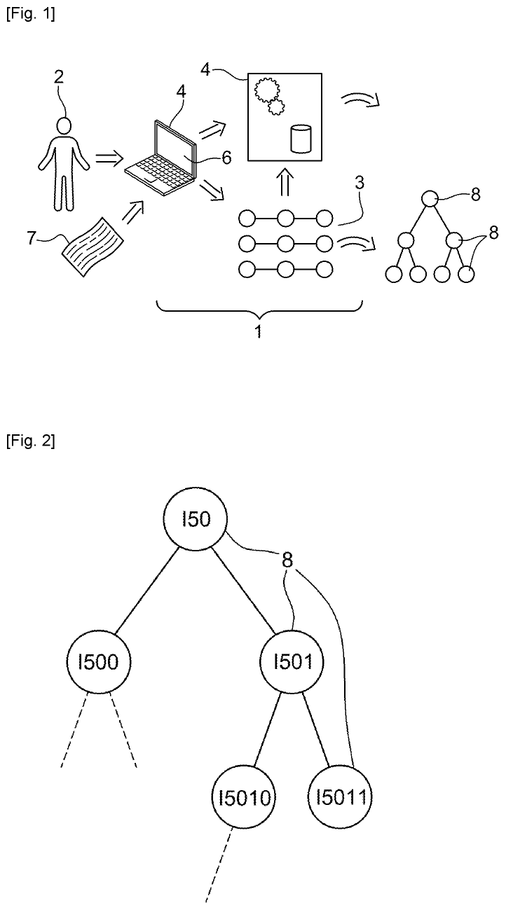

FIG. 1 shows the elements used when implementing the method. This method is implemented using a software program 1 controlled by a user 2 by means of a computer. This software program proposes a user interface 6 via which the user 2 controls the invention. This software program is the interface between the user 2 and all the other elements. This software program controls in particular a self-learning model 3 designed to perform automatic classifications. Under the control of the software program 1, the model 3 supplies proposals of results of these classifications, associated with scores, to automated means 4.

The automated means 4, called indifferently throughout the application “calculating means”, “computer calculating means”, or “automated means”, designate any type of computer means, in particular calculating means and communication means. They therefore implement processors, databases and communication networks. These means 4 can be grouped together or separated, and can operate remotely. They are designed to use the results of the classifications supplied by the model 3 to generate evaluations and justifications to be displayed to the user 2.

The data 7 supplied as input to the software program 1 are a list of medical stays, the determined data supplied as output by the software program via the automated means are codes 8, corresponding to medical diagnoses assigned to at least some of these stays, and associated with justifications.

B) The Data

The data 7 forming a medical stay, or hospital stay, include all documents concerning the patient or their hospital stay. The data may consist in particular of modes of entering and leaving a medical unit, dates of the stay, hospitalisation report, medical correspondence, imaging or examination reports. The data may also consist of the “standardised discharge summary” (SDS) containing all the information of the “medical unit summary (MUS) concerning the stay. Each MUS includes for example a FINESS number of the hospital, the SDS number, a stay administrative number and a diagnosis related group (DRG) number. It may also contain “common classification of medical acts” (COMA) codes. Other data, such as any data related directly or indirectly to the patient's stay, may also be used. These data 7 are supplied as input to the self-learning model 3. In more concrete terms, they are supplied as vectors. Thus, amongst these data 7, the administrative data include numerical and categorical characteristics which are converted into vectors. The textual data are also extracted, concatenated and vectorised, for example using the Scikit-learn library. The two vectors, that of the administrative data and that of the textual data, are then concatenated to form a single vector corresponding to the stay.

The output data 8 are codes. They correspond to the International Classification of Diseases (ICD) code of the main diagnosis of the MUS, the ICD code of the diagnosis related to the stay, if any, and ICD codes of the related diagnoses, if any. Code I48, for example, corresponds to “atrial fibrillation and flutter”, code I48.2 to “chronic atrial fibrillation”. Code D61.1+Y43.3 corresponds to a “drug-induced aplastic anaemia” as main diagnostic, and to an “undesirable effect of chemotherapy treatments during therapeutic use” as associated diagnosis. The self-learning model 3 must therefore assign each stay 7 supplied as input to this type of code 8.

C) Tree-Structure Results

The PMSI classification can be illustrated as a tree structure, as shown on FIG. 2. Some codes correspond in fact to generic diagnoses, others to more precise diagnoses and others to even more precise diagnoses. For example, code I50 corresponds to a heart failure, code I500 to a congestive heart failure, and code I501 corresponds to a left ventricular failure. Then code I5010 corresponds to a left ventricular failure with left ventricular ejection fraction higher than or equal to 50, while code I5011 corresponds to a left ventricular failure with left ventricular ejection fraction less than 50 and greater than or equal to 40. We may therefore speak of a hierarchy between parent nodes corresponding to general diagnoses and child nodes associated with more precise diagnoses.

The classification results may correspond to any code, irrespective of its hierarchic level in this tree structure. Preferably, however, the code should correspond to the lowest possible hierarchic level, in other words to the most precise diagnosis possible. The pricing of the medical activities may in fact change at every level.

We will now describe the operation of an example of a self-learning model, model 3, used to automatically classify medical stays 7 within diagnosis classes 8. We will then describe the operation of the invention as such, in other words the method for evaluating the results proposed by the self-learning model.

II. The Classification

A) Learning

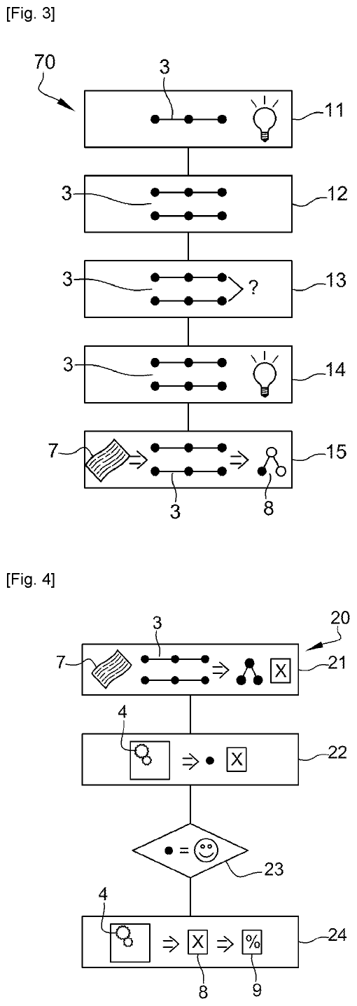

Model 3 is configured using a method 70 shown on FIG. 3.

One type of suitable classification model is a multilayer perceptron, which is the type chosen for model 3. Note that the purpose of a self-learning model is to assign to a set of input data—in this case, data concerning a medical stay 7—an output class—in this case a PMSI code 8. To do this, the model 3 is trained, in step 11, on a learning database where the stay codes supplied are already known. The training objective is to minimise the cross entropy. This objective is achieved using the gradient descent algorithm. This model is implemented and trained using Tensorflow, for example.

After learning, the aim is to optimise and calibrate the model 3. Other “validation data” are used, for which once again the output classes are known, to specify the “hyperparameters” of the model: its number of neurons, axons, and generally all the characteristics defining the learning model. This step 12 aims in particular to prevent overlearning of the model 3. In addition, to calibrate the model, the Temperature Scaling algorithm, described in “On Calibration of Modern Neural Networks” (Guo et al, 2017), is used. This calibration performed in step 13 enables the model 3 to associate a score representing a precision level with the output class 8. This score may for example be a numerical value between 0 and 1. Thus, for a given stay 7, a first class can be predicted with any score, and a second class with a higher score. If the model has been well calibrated, then the second class is more likely to be correct than the first.

Lastly, in step 14, other data with known classes are used to test the ability of the model 3 to generalise.

After these steps 11 to 14, the model is correctly trained to predict the output class 8 of medical stays 7 supplied as input, in other words it is capable of assigning them one or more PMSI codes. A code may for example be “a main diagnosis”, which can be associated with one or more codes of associated diagnoses, codes corresponding to CCMA acts, or even a code predicting the patient's DRG group.

B) The Proposals

The automatic classification is therefore described below, according to step 15. During the classification phase, the user 2 supplies medical stays 7 to the self-learning model 3. The model then supplies as output a list of results 8: at least one PMSI code and a numerical score associated with this result. The model may sometimes supply several code-score pairs for a given stay. In the invention, these results are then evaluated, which we will describe in detail below.

III. Evaluating the Confidence Level

A) Determining the Confidence Level

The object of the invention is to propose to the user, in a first embodiment, the results for which the associated confidence level 9 is higher than a given threshold. We will first describe how to calculate this “confidence level” 9, also called “precision”, using a method 20 shown on FIG. 4.

This level 9 is determined by training the automated means 4 on a test database. The steps are shown on FIG. 4. In a step 21, for medical stays 7 which have known classes, the model 3 outputs result-score pairs. Then, in step 22, the automated means 4 select, for each stay, the result with the highest score X. Note that this result is a class, in other words a PMSI code. The selected result is then compared with the expected result in step 23. By performing this type of classification/comparison on a sufficiently large sample of test data, the automated means determine, in step 24, firstly a correlation between the score X and the selected result, and secondly whether or not the selected result is accurate. As an example, we will take one hundred classifications for which the result is class I48 with the score 0.65 supplied by the calibrated model. If out of these one hundred classifications, 90 classified stays actually correspond to class I48, then the automated means deduce that the confidence associated with any result concerning class I48 with a score of 0.65 is 90%.

In this way, the automated means 4 therefore learn how to determine the confidence levels 9 associated with the results, by using the scores that have already been determined by the self-learning model 3 and supplied to the automated means 4. Thus, for each result 8 supplied by the model 3 with its score, the automated means 4 can determine a confidence level 9 as a percentage, also called “precision”. This precision 9 corresponds statistically to the probability that the result 8 is correct, in other words that the PMSI code proposed actually corresponds to the stay 7 supplied as input.

B) The Confidence Threshold

Using the software program 1, the user 2 can choose an “automation” confidence threshold, above which the results are automatically classified by the software program 1, without having to be validated by the user. This threshold may be 99% for example. This means that, for results whose precision is at least 99%, the stays are automatically classified as predicted by the self-learning model 3. The user can obviously set this threshold as required.

In this case, we are mainly interested in the “quasi-automation” threshold. The user 2 can in fact set a confidence threshold so that only those results whose calculated precision is higher than or equal to this “quasi-automation” confidence threshold, but less than the “automation” confidence threshold, are displayed on the user interface 6. This therefore concerns stays 7 for which the classification is probably correct, since the confidence level of their results 8 is higher than or equal to the quasi-automation confidence threshold, but far from being certain, since this confidence level is less than the automation confidence threshold.

The user 2 can validate these results proposed by the model 3, in other words assign the diagnosis code proposed by the model to the stays concerned. Thus, the user 2 only has to check whether the code 8 proposed seems to correspond to the medical stay 7 supplied as input and simply click to validate or reject this proposal.

IV. Evaluating the Justifiability Level

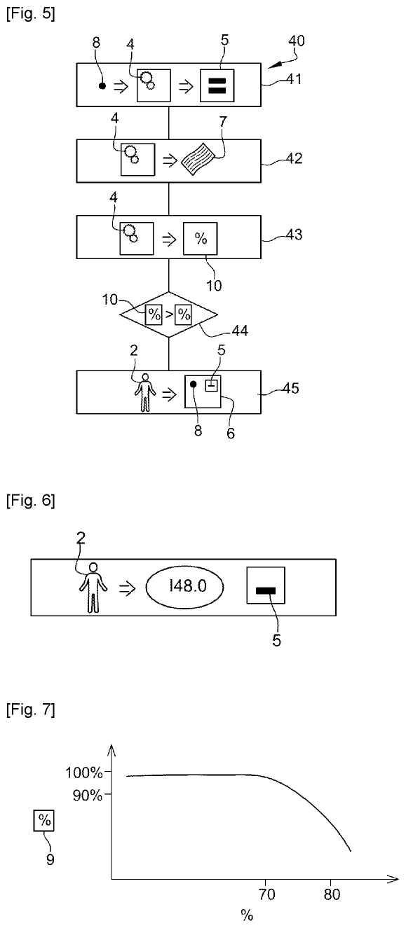

In a second embodiment shown on FIG. 5, the evaluation does not only concern the confidence level 9 of a proposed result. The automated means 4 also determine a justifiability level 10 associated with each result proposed, and only propose to the user 2 the classification results of the model 3 for which the confidence levels 9 and the justifiability levels 10 exceed respective predetermined thresholds.

As shown on FIG. 5, in step 41, the means 4 first read a list of justification data 5 to be detected for each possible result 8. Then, for each stay 7 supplied as input, for which the classification result is proposed to the user, in step 42, the means detect amongst the data 7 the data in the previously created list 5 which will be used to justify the result 8 to the user 2. In step 43, the means 4 determine a justifiability level 10 using the detected justification data. Lastly, in step 44, the means check whether the justifiability level 10 is higher than or equal to a predetermined justifiability threshold, configured by the user 2. If this is the case, and if the confidence level of the result is higher than the confidence threshold, the result is displayed in step 45 on the user interface 6, together with justification data detected for this result.

We will now describe each of these steps.

“Justifiability level” means a quantity and/or a quality of data determined by the automated means 4 in order to justify the results proposed to the user 2. As an alternative, however, it could be any way of quantifying the ability of the means 4 to justify the results proposed 8 to the user 2.

A) The Justification Data

In this case, for a given stay 7 and amongst all the data relating to said stay, the automated means detect the “justification” data 5 corresponding to the proposed result. Thus, for a patient suffering from chronic atrial fibrillation, the means may for example detect a sentence in a hospitalisation report clearly mentioning a “chronic atrial fibrillation” or similar symptoms. If the classification result of this stay is a code including “I48.2” (PMSI code corresponding to this type of fibrillation), then the sentence is proposed to the user at the same time as the result, in order to justify the result. Thus, the user 2 can not only validate results for which the confidence level is higher than threshold predetermined by the user, but also use the justification data 5 supplied to decide whether or not to validate this result, rather than search in the medical record of the stay concerned.

B) Determining the Justification Data

To detect the justification data, such as the sentence mentioned above, the means 4 must know them, which corresponds to step 41 which we will now describe. Thus, in a first variant shown on FIG. 6, the means a priori know the data to be detected 5, associated with all or some of the possible results. These data may have been supplied beforehand to the means 4 by the user 2 or by another human means. It may be for example a list of terms associated with each possible result. For example, as shown, a user may have supplied a list of justification data 5 for code I48.0 corresponding to a “paroxysmal atrial fibrillation”, this list containing the terms: “atrial fibrillation”, “acfa”, “fa”, or terms relating to the therapeutic treatment of this diagnosis, such as the term “cordarone”.

In a second variant shown on FIG. 7, the automated means 4 have previously implemented a learning method in order to determine the justification data 5 which they must detect afterwards. This learning is carried out on a learning database, in other words in which the classes corresponding to the stays are known.

Note that a stay 7 supplied as input consists of a vector corresponding to several administrative data and several textual data.

In a step 31, model 3 is used to classify several stays concerning the same class, for example stays concerning a ventricular tachycardia, therefore code PMSI I47.2. The means determine the confidence levels associated with each result. We will assume for example that they oscillate between 85 and 95% precision depending on the stays. In a step 32, the means 4 select amongst them the stays which are correctly classified, in other words those for which the model 3 has correctly assigned code I47.2, or a code including code I47.2. In a step 33, one or more data of the vector 7 supplied as input of the model are masked. For example, the vector is recreated using the same administrative and textual documents, but deleting all the occurrences of the term “ventricular”. In a step 34, new classifications of the same model 3 are made on these vectors. The means 4 then determine the confidence levels associated with each result.

In a step 35, the new confidence levels are compared with the old ones. If, on average, the confidence levels have decreased drastically, for example by 5%, this means that the masked data, in this case the term “ventricular”, is particularly important in the choice of the model 3 to assign code I47.2 to these stays. This is why, in step 36, the automated means 4 then add the masked data, in this case the term “ventricular”, to a list of justification data 5 corresponding to class I47.2. This method can be applied to any class, generally to any possible result. Obviously, the drop in confidence level from which a data is considered as being a justification data can be configured by the user 2.

Obviously, the two variants described previously so that the means 4 know the justification data 5 to be detected can be implemented simultaneously: some justification data may have been supplied manually, while other data may have been “learned” by the automated means 4.

Thus, each possible classification result, in other words each possible PMSI code, is associated with a list of justification data in this step 41.

As an alternative, in an embodiment not shown, the justification data to be detected can be determined a posteriori, in other words after the model 3 has classified the stays 7. In this case, steps similar to steps 31 to 36 are performed not for a series of training stays corresponding to a given class and on a learning database, but for each stay 7 corresponding to a result 8, and therefore on “real” data, without prior learning. Thus, after performing the classification, for each stay 7, the means 4 mask some data of the stay between successive classifications of these data, in order to determine which of the data cause(s) a substantial variation in the calculated confidence level. These data are therefore considered as justification data justifying the result 8.

This a posteriori detection method is less precise than the method 30 performed previously on a learning database and described above, since it can be used to average the confidence levels for several stays corresponding to a given output class, and therefore to determine the most relevant types of data to be detected for all types of stay. However, this a posteriori detection method can supply justification data if the method 30 has not been implemented beforehand for some or even all of the results.

Obviously, this method may also be combined with the preceding methods performed before the classification.

C) Determining the Justifiability Level

The justifiability level 10 can be defined in various ways. It may correspond to a number, for example to a percentage expressing the number of justification data on the list detected in the data 7 of a stay to be classified. It may also be a scale, with four levels four example: good, average, low, absent. In this case, each list of justification data is organised according to these levels. The “good” level includes some highly relevant terms, for example the exact title of the diagnosis. If one or more terms on this relevant list are detected, the justifiability level is good. The “low” level includes less relevant terms on the list of justifications, for example terms which may concern several diagnoses. The “average” level corresponds to the detection, for a given stay, of terms belonging to the “good” category and other terms belonging to the “low” category.

These lists can be organised manually according to these levels, by a user indicating the particularly relevant terms that can be used to justify a result satisfactorily, as well as the terms that can be used to justify a result, but with less certainty.

This organisation may also be carried out by the automated means 4, especially when they “learn” how to detect the justification data 5 (see above). For example, if a data masked during this learning causes the confidence level, associated with a result, to drop by 10%, then this term can be assigned to the “good” category. Obviously, these thresholds aimed at organising the justifiability levels can be configured by the user 2.

Thus, for each result 8 proposed to the user, a justifiability level 10 of this result is determined by the automated means 4. Then, depending on this level, the result is proposed or not by the means 4 to the user 2. The user can in fact configure a justifiability threshold, below which the corresponding results will not be proposed to the user 2.

In a third embodiment, we move from step 42 of FIG. 5 to step 45 without going through steps 43 and 44. In other words, the means detect the justification data but do not determine a justifiability level. This amounts to configuring the justifiability level in “absent” mode. All the results proposed to the user are those for which only the confidence level exceeds a predetermined threshold, and all are associated with justification data if they are detected.

D) Summary of the Thresholds

In short, once the model 3 has classified stays 7, the automated means 4 evaluate the results, in other words the codes 8 proposed to these stays. They first determine the confidence levels associated with these results. Those for which the result is higher than an automation threshold are classified automatically. For these levels, in fact, the classification error rate is too low compared with the PMSI encoding quality requirements. This automation threshold is for example 99%, which means that statistically 1% of the stays should be incorrectly classified.

Those for which the confidence level is too low, in other words below the quasi-automation threshold, are removed from the process. They will be processed separately, for example by studying the medical record of the stay 7. For those which exceed this threshold, the automated means 4 determine the justifiability level associated with each result. Those for which the justifiability level is less than the predetermined justifiability threshold are excluded and will be processed separately. We are therefore left with the results corresponding to confidence and justifiability levels higher than the thresholds. These results are proposed to the user 2 with the justification data detected by the means 4 and associated with each result. The user can decide to validate each of the results, in this case the classification is final, or on the contrary reject some.

In other words, by evaluating the results which are not “automated”, the classification proposals can be filtered according to two cumulative criteria: only the results which are higher than or equal to the “quasi-automation” confidence threshold and which, at the same time, are higher than or equal to the justifiability threshold, are proposed to the user, who can predetermine the two thresholds.

Note that, even if the justifiability level is not determined, the justification data can be detected and displayed to the user. This corresponds to the justifiability threshold configured in “absent” mode.

V. The User Interface

The user interface 6 schematises what the user 2 displays on a computer screen when implementing the invention. The progression of this interface is shown on FIG. 9.

A) The Hierarchic Level of the Results

As described above, the results are organised as a tree structure, with results at different hierarchic levels. Using this tree structure, the user can make requests to the various parameters.

The user can first decide in which hierarchic levels they want to perform the classification, in step 51. The stay does not always have to be assigned to the most precise code. The user can therefore configure the model 3, via the interface, so that the results are more generic, which also makes the results more precise.

B) Configuring the Confidence Threshold

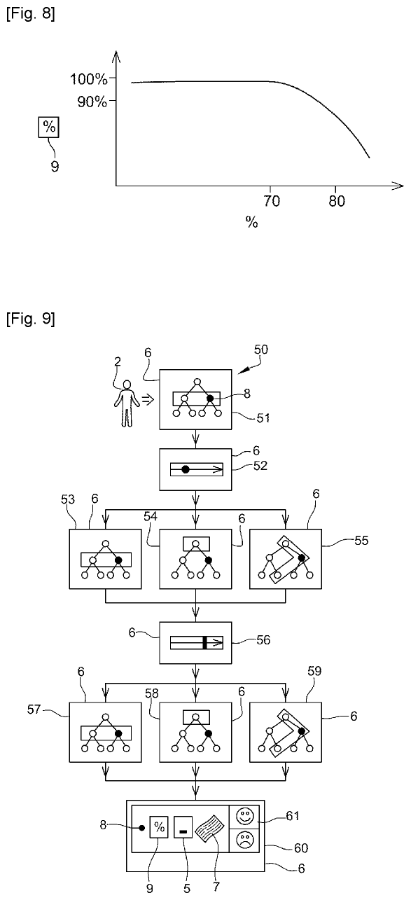

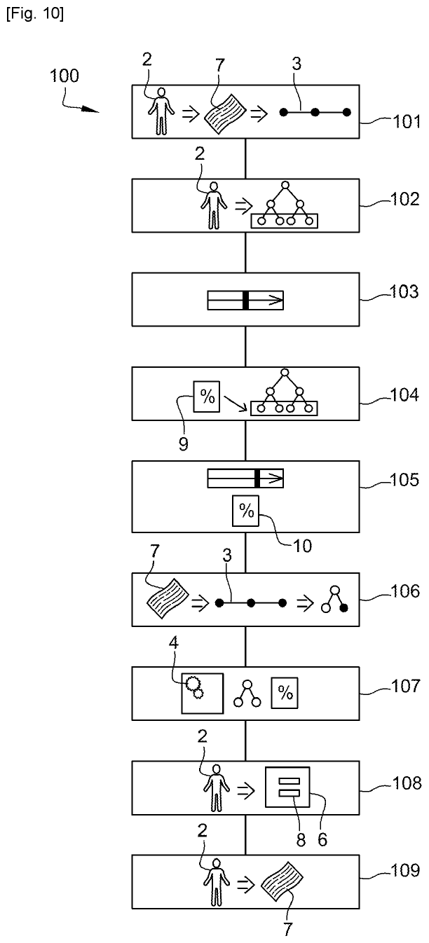

To configure the confidence threshold, the user 2 can use precision-sensitivity curves, as shown on FIG. 8. These curves are produced beforehand, during a large number of test classifications, with one curve per class, in other words for each possible result or code. On the x-axis, they represent the sensitivity, in other words the ratio between the number of stays predicted in the class and the total number of stays which should be predicted in this class. On the y-axis, they represent the precision, which corresponds to the confidence level 9 described previously. In other words, this is the number of stays correctly predicted out of the total number of stays predicted.

Using these curves, in step 52 the user can choose, for each class, the precision threshold, in other words the confidence threshold, above which the user wants the results to be displayed. This curve means that the higher this threshold, the higher the probability that the results proposed to the user will be correct, but the lower the number of results proposed. The user must therefore choose between precision and the number of proposals to be checked.

The user also uses this interface 6 to set the automation threshold above which the results are automatically validated.

In a first variant, these thresholds correspond directly to the hierarchic level of the results proposed. In other words, the automated means 4 check whether or not the precision associated directly with the result exceeds the confidence threshold.

In a second variant shown in step 53, the confidence level determined does not correspond to that associated directly with the result, but to a parent node of the result within the tree structure. The user 2 can therefore configure how to determine the confidence level. For example, we will assume that the quasi-automation confidence threshold configured by the user is 80%. We will also assume that the result proposed after classifying a stay is code I481 (“persistent atrial fibrillation”) with a precision of 75%. In the first variant, the confidence level of the result proposed was compared with the threshold, so in this case the result would not be submitted to the user.

However, in the second variant shown in step 54, the confidence level corresponding to code I48 (“Atrial fibrillation and flutter”), which corresponds to the parent node of the node associated with the result, is compared with the threshold. The precision is probably higher than, for example 85%, since this node corresponds to a more generic diagnosis. In this second variant, the result I481 will therefore be proposed to the examiner since the confidence level associated with the parent of this result exceeds the confidence threshold.

The user can therefore configure as required the hierarchic level of the results to be proposed, but also and independently, at which hierarchic level the confidence associated with the result is determined. Obviously, the confidence is necessarily associated with a level that is equal to or more generic than that of the result.

In a third variant shown in step 55, the user 2 can decide that the measured precision corresponds to a group of nodes, and not to a single node. For example, if the threshold is 80%, the user may want to determine all the stays which are assigned, with at least 80% precision, to one of the codes of a group consisting of code I44.4 (left anterior fascicular block), I44.5 (left posterior fascicular block), and I44.3 (left bundle-branch block). If a determined confidence level is 85%, it is displayed to the user since this confidence is higher than the 80% threshold, and this precision means that there is an 85% chance that this stay does in fact correspond to one of these three codes. This therefore represents a way of determining which stays correspond to only some diagnoses which are of interest to the user.

C) Configuring the Justifiability Threshold

Using the same interface, the user 2 can choose the justifiability threshold in step 56. As seen previously, the justifiability level may correspond to a numerical score, in which case the user can configure the numerical value of the threshold. It may also correspond to a scale level, such as “good”, “average, “low” or “absent”. In this case, the user sets the threshold above which the associated results are to be proposed. If the user chooses the “absent” threshold, only the confidence level of the results will be taken into account, not the justifiability.

Determination of the justifiability level can be adapted in the same way as that of the confidence level. Thus, it may correspond directly to the result proposed, as shown in step 57. In a variant shown in step 58, it may also correspond to a parent node of the proposed result. Lastly, it may correspond to a justifiability level associated with a group of possible results, as shown in step 59.

Thus, the two thresholds are set by the user, and associated with the result, to a hierarchically higher node or to a group of nodes, independently of the result and independently of each other.

C) Self-Adaptive Setting

In an embodiment variant not shown, self-adaptive setting of one or both thresholds can be planned. This means that instead of unique thresholds, the thresholds and hierarchic levels they concern will change depending on the confidence level associated with the result. We will take the example of a self-adaptive confidence threshold which is set to 80% as a first approximation and for a particular hierarchic level. If the confidence level associated with a classification result, for this hierarchic level, is not obtained, the automated means 4 will then check whether the confidence threshold has been reached for a parent node of the result. They may also modify the confidence threshold to be reached for this parent node. If the threshold is reached, then the result corresponding to the parent node can be displayed with the associated precision. This example is not limiting, the self-adaptive setting can be performed in different ways. Its purpose is to display results for each predicted stay, but results that are as precise as possible. If a precise result is impossible for some stays, then this type of setting can be used to display to the user an output class that is broader than required, but which gives the user an indication of the stay classification.

With this variant, if the results displayed are not suitable, the user does not have to redefine the thresholds and hierarchic levels they concern, but simply rely on a single evaluation of all the stays, and for which the confidence levels will be adapted to the results of each stay.

D) Validating the Choices

After setting the thresholds and the way in which the confidence and justifiability levels will be determined, in step 60, the user can start the evaluation, by the automated means 4, of the results proposed by the model 3. A list of results 8, which the user can decide to validate or reject, is then displayed on the user interface 6.

Note than on a given page, all the results, in other words all the classifications proposed, can be displayed to the user, who can scroll through these proposals as required. In other words, the results page displays a list of stays supplied as input to the model and all the codes predicted by the model for each stay. The user can therefore scroll quickly through all the results for all the stays, and will not have to move from one stay to another to find the classification results of these stays. As an alternative, the software program 1 can also propose the results corresponding to a single stay per page.

These results are those for which the precision (confidence) levels 9, and optionally the justifiability levels 10, are higher than or equal to the thresholds configured by the user.

For each result on the list, the interface displays to the examiner a reference to the medical stay 7 supplied as input to the model 3, the confidence or precision level 9 associated with the result of this stay, whether it is the level associated directly with the result or the level associated indirectly with the result by reflecting the precision level associated with a hierarchically higher node or to a group of nodes. The justifiability level 10, if the user wishes to know it, can also be displayed, once again whether it is associated directly or indirectly with the result. Lastly, the justification data 5 detected by the means 4 are also displayed.

Thus, for each result 8 on the list, the user 2 can quickly check whether this result 8 seems to be correct by glancing at the justification data 5 displayed. The user therefore does not have to search in the medical record associated with the stay 7 and can simply click the button 61 to validate the proposed result.

As an alternative, the results 8 can already been displayed as “validated” so that the user 2 only has to “reject” them if deemed necessary in view of the justification data supplied.

VI) Implementation

A) Main Implementation

To summarise the means and steps implemented, we will now describe a method of implementing the invention by the user 2, in reference to the method shown on FIG. 9.

The user 2 wants to classify a list of medical stays as precisely as possible and as quickly as possible, in other words assign one or more PMSI codes to each one. These codes are grouped as a tree structure.

In step 101, the user supplies to the model 3 the list of medical stays to be classified. In step 102, the user configures the hierarchic level of the results to be proposed on screen. In step 103, the user sets the predetermined confidence threshold. In step 104, the user configures how to determine the confidence level 9 to be compared with the threshold set in step 103. This level could be a confidence level of the result itself, or corresponding to a parent node of the result, or to a group of nodes. In step 105, the user performs the same settings for the justifiability level.

In step 106, the user starts the classification, performed by the model 3. In step 107, the user starts the evaluation, performed by the means 4, of this classification.

In step 108, the results are displayed on the user interface. Only the results respecting the confidence and justifiability levels are displayed, together with justification data. The user 2 can then validate or reject the classification of these stays, in other words the codes assigned to these stays.

The main advantage of the method described previously is that the number of stays to be processed manually is considerably reduced by evaluating “quasi-automatable” stays. As a corollary, the number of these stays is related to the confidence and justifiability thresholds configured by the user: for example, the lower the precision requested, the fewer the number of stays removed from the validation phase. The user can therefore decide at any time on the maximum error rate that can be accepted and the time to be spent performing an in-depth study of the stays.

Step 109 concerns the stays 7 which have not been classified since they did not respect the confidence or justifiability thresholds, as well as those which respected them but for which the user 2 rejected the results.

Even for these stays to be classified manually, in fact, the means 4 provide valuable help. The user can therefore be helped by the result proposed by the classification, or even by the justification data determined. Even if the proposed result is incorrect, in fact, this information is highly relevant since it can encourage the user for example to assign to the stay a code close to that proposed, without having to perform an in-depth study of the medical record of the stay.

The evaluation method described therefore saves a considerable amount of time when assigning PMSI codes to medical stays.

B) Quality Control

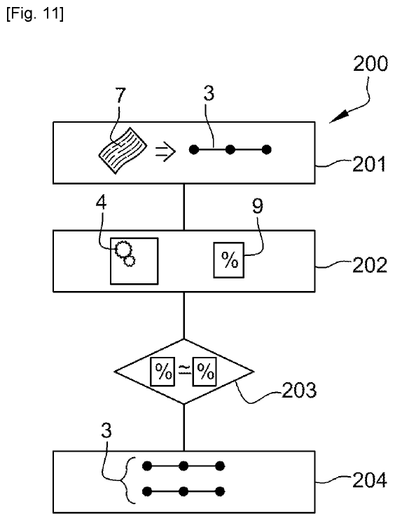

In a second embodiment shown on FIG. 11, the means described previously can be used to control the classification quality of a classification model, for example model 3. Databases of test medical stays, in other words whose PMSI codes are already known, are used to do this.

In a step 201, the model 3 classifies these medical stays for which the codes are a priori known. In step 202, the automated means 4 evaluate the classifications as described previously, and indicate in particular the precision of each result supplied.

Since the correct codes are known, the results and their precision can be compared with the expected results. This comparison is performed by the automated means 4 in step 203, which can thus deduce whether the differences found suggest that the classification model is not satisfactory or whether the error rates of this model comply with the precision levels determined by the means 4. In step 204, the user can then decide to modify the model 3 to make it more efficient. These modifications can be performed manually or automatically.

In addition, the automated means 4 can be used to check why some stays have been classified incorrectly, in particular by examining their associated justification data.

The invention described above relates in particular to a method 100 for evaluating results of an automatic classification of medical data 7, comprising the implementation of the following steps:
computer calculating means 4 perform an automatic classification of one or more medical data 7,
the computer calculating means 4 determine a confidence level (9) associated with at least one result 8 of the classification,
the computer calculating means 4 compare the confidence level 9 associated with the result with a predetermined confidence threshold, and
the computer calculating means 4 display the result to a user 2 if the confidence level associated with the result is higher than or equal to the predetermined confidence threshold.

This same method can be considered to be an automatic classification method, specific in that it comprises an evaluation step. It can therefore be reformulated as follows, for example: method for automatically classifying medical data, comprising the implementation of the following steps:
computer calculating means perform an automatic classification of one or more medical data,
the computer calculating means determine a confidence level associated with at least one result of the classification,
the computer calculating means compare the confidence level associated with the result with a predetermined confidence threshold, and
the computer calculating means display the result to a user if the confidence level associated with the result is higher than or equal to the predetermined confidence threshold.

Lastly, this method may also be considered to be a method for generating structured data from unstructured or structured data, in which automatic classification proposals are made for medical data, the method comprising an evaluation phase, at least one proposal result, wherein:
automated means determine a confidence level associated with the result,
the automated means compare the confidence level associated with the result with a predetermined confidence threshold, and
the automated means display the result to a user if the confidence level associated with the result is higher than or equal to the predetermined confidence threshold.

The invention is not limited to the embodiments described and other embodiments will be clearly apparent to those skilled in the art.

Thus, apart from words or sentences, the medical data to be classified can be of any type, for example quantitative. The data could be the patient's body mass index, tobacco consumption in number of packets per year, the volume of a haemorrhage, the volume of a urinary retention, the left ventricular ejection fraction, etc.

In addition, the invention is not limited to the prediction of PMSI codes. For example, instead of the main and associated diagnoses concerning medical stays, the prediction of COMA acts can be predicted and therefore evaluated, using the same data as those supplied as input to predict PMSI codes. Once again, therefore, these predictions can be evaluated and/or justified using the invention.

Note that the medical data correspond to any data relating directly or indirectly to the medical field, and not necessarily to data resulting from a medical stay in a healthcare establishment. Thus, they may correspond to medical elements outside the field of PMSI encoding. For example, the method may concern the classification of a patient's status with respect to a given genetic mutation, or with respect to a type of allergy. These are still medical data, but of a broader nature than PMSI encoding data. The classification may also concern the medical or surgical history of a patient. It may even concern the patient's family history. In this case, the data does not necessarily concern a medical stay, the aim is not necessarily to predict a PMSI code or a diagnosis, but we are still dealing with the classification of medical data.

Note also that the invention does not relate to a classification model or to a particular type of self-learning model. The data supplied for the evaluation are only the result of a classification and a score associated with this result, and these data do not depend on the classification model. The invention can therefore be adapted to all types of classification model.

Generally, the invention is not limited to automatic classifications of medical data. In concerns in fact the evaluation of the results proposed after classification, it does not depend on the types of data and can therefore be extended to any type of field where automatic classifications are performed.

1. Method (100) for evaluating results of an automatic classification of medical data (7), comprising the implementation of the following steps:
computer calculating means (4) perform an automatic classification of one or more medical data (7),
the computer calculating means (4) determine a confidence level (9, 20) associated with at least one result (8) of the classification,
the computer calculating means (4) compare the confidence level (9) associated with the result with a predetermined confidence threshold, and
the computer calculating means (4) display the result to a user (2) if the confidence level associated with the result is higher than or equal to the predetermined confidence threshold.

2. Method (100) according to claim 1, wherein the computer calculating means (4) also display to the user at least one detected justification data (5) justifying the result (8).

3. Method (100) according to claim 2, wherein the justification data (5) belongs to the one or more medical data (7) for which the classification performed led to the result, and is textual.

4. Method (100, 40) according to claim 1, wherein:
the computer calculating means (4) also determine a justifiability level (10) associated with the result (8),
the computer calculating means (4) compare the justifiability level (10) associated with the result with a predetermined justifiability threshold,
the computer calculating means (4) display the result to the user if, in addition, the justifiability level (10) is higher than or equal to the predetermined justifiability threshold.

5. Method (100, 40) according to claim 1, wherein, to determine the justifiability level (10) associated with the result (8):
amongst the one or more medical data (7), the computer calculating means (4) detect at least one or one of the justification data (5) justifying the result (8), and
depending on the or each justification data (5) detected, the computer calculating means (4) determine the justifiability level (10) associated with the result.

6. Method (100, 30) according to claim 2, wherein, to detect a justification data (5) justifying the result (8), the computer calculating means (4) first determine one or more types of justification data (5) to be detected.

7. Method (100, 30) according to claim 6, wherein, to determine one or more types of justification data (5) to be detected, from a learning medical database:
using learning medical data from the database, the computer calculating means (4) perform a first classification and determine a first confidence level associated with a result of the first classification,
the computer calculating means (4) mask at least one of the learning medical data used to perform the first classification,
the computer calculating means (4) perform a second classification using data not including the masked learning medical data and determine a second confidence level of the second classification,
if the difference between the first and second confidence levels is higher than a predetermined threshold, the computer calculating means (4) record a type of the masked learning medical data as type of justification data to be detected.

8. Method (100) according to claim 2, wherein, to detect a justification data justifying the result (8), one or more types of justification data (5) to be detected are supplied to the computer calculating means (4) by the user.

9. Method (100) according to claim 2, wherein the computer calculating means (4) detect the one or more justification data (5) using the one or more medical data (7) for which the automatic classification performed led to the result (8).

10. Method (100) according to claim 9, wherein, to detect the one or more justification data (5) using the one or more medical data (7) for which the automatic classification performed led to the result (8), since the classification is a first classification and the confidence level associated with the result is a first confidence level:
the computer calculating means (4) mask the or at least one of the medical data used to perform the first classification,
the computer calculating means (4) perform a second classification using the data not including the masked medical data and determine a second confidence level of the second classification,
if the difference between the first and second confidence levels is higher than a predetermined threshold, the computer calculating means (4) record the masked medical data as detected justification data.

11. Method (100) according to claim 1, wherein the result (8) belongs to a tree structure of nodes corresponding to possible results.

12. Method (100) according to claim 11, wherein the confidence level (9) is associated with a node of the tree structure which is a parent of a node corresponding to the result (8).

13. Method (100) according to claim 11, wherein the confidence level (9) is associated with a group of nodes of the tree structure comprising a node corresponding to the result (8).

14. Method (100) according to claim 4, wherein the justifiability level (10) is associated with a node of the tree structure which is a parent of a node corresponding to the result (8).

15. Method (100) according to claim 4, wherein the justifiability level (10) is associated with a group of nodes of the tree structure comprising a node corresponding to the result (8).

16. Method (100) according to claim 12, wherein, depending on the confidence level associated with the result, the computer calculating means (4) modify the association of the confidence level and preferably the confidence threshold.

17. Method (100) according to claim 14, wherein, depending on the justifiability level associated with the result, the computer calculating means (4) modify the association of the justifiability level and preferably the justifiability threshold.

18. Method (100) according to claim 1, wherein the computer calculating means (4) allow a user (2) to predetermine the confidence threshold of claim 1 and preferably the justifiability threshold of claim 4.

19. Method (100) according to claim 1, wherein the computer calculating means (4) allow the user (2) to reject the result (8).

20. Method (100) according to claim 1, wherein the or at least one of the medical data (7) concerns at least one medical stay of at least one patient in a healthcare establishment.

21. Method (100) according to claim 20, wherein, since several medical data (7) concern several medical stays, the computer calculating means (4) display to the user the classification results (8) of all the stays (7).

22. Method (100) according to claim 1, wherein the classification result (8) corresponds to one or more codes concerning medical diagnoses, acts or procedures, Diagnosis Related Groups (DRG) or Homogeneous Groups of Stays (HGS).

23. Method (100) according to claim 1, wherein the confidence level (9) associated with the result is a numerical value associated with a probability that the result is correct.

24. Method (100) according to claim 23, wherein, to determine the numerical value associated with a probability that the result is correct, using a learning medical database:
the computer calculating means (4) classify learning data;
for each classification, the computer calculating means determine primary numerical values associated respectively with classification results;
for each classification, the computer calculating means select the classification result having the highest numerical value;
for each classification, the computer calculating means compare the selected result with a correct expected result; and
depending on the comparison results, the computer calculating means (4) determine the numerical value associated with a probability that the selected result is correct.

25. Data processing system (1, 3, 4, 7) comprising means for implementing the steps of the method (100) according to claim 1.

26. Computer program, comprising instructions which, when the program is executed by a computer, instruct the computer to implement the steps of the method (100) according to claim 1.

27. Method of obtaining the program of claim 26 in order to download it on a telecommunication network.

28. Computer-readable data medium, on which the computer program according to claim 26 is stored.
